(19) United States
(12) Patent Application Publication
(10) Pub. No.: US 2018/0103766 A1
(43) Pub. Date: Apr. 19, 2018

(54) Lumbar or Neck Support With Beverage Bottle

(71) Applicant: Junliang Ou, Alhambra, CA (US)

(72) Inventor: Junliang Ou, Alhambra, CA (US)

(21) Appl. No.: 15/728,031

(22) Filed: Oct. 9, 2017

Related U.S. Application Data
(60) Provisional application No. 62/407,797, filed on Oct. 13, 2016.

(51) Int. Cl. A47C 7/62; A47C 7/42; A47C 7/38; B65D 1/02; B65D 81/38; A61F 7/08; A61F 7/10

(52) U.S. Cl. CPC A47C 7/62; A47C 7/425; A47C 7/383; A61F 2007/0027; B65D 81/3876; A61F 7/086; A61F 7/103; B65D 1/0223

(57) ABSTRACT

The present invention is (1) a lumbar support and/or (2) a neck support incorporating a water or liquid containing bottle for beverages. The invention devices are shaped to provide lumbar or neck support but retain a removable cap and a large internal volume for storage of a beverage.

This application claims priority to Provisional Application Ser. No. 62/407,797, filed on Sep. 13, 2016, the contents of which are hereby incorporated by reference into the present application.

### FIELD OF THE INVENTION

The present invention is broadly directed to lumbar and neck supports provided to a user to supplement portions of seating for a person along a back rest.

### BACKGROUND OF THE INVENTION

Millions of Americans suffer from back pain which generally involves the cervical and lumbar spines. The majority of the back pain is related to spondylosis or what is generally called degenerative disease. Other contribution factors are related to poor posture, congenital or developmental back problems and prior history of back injury. Back supports provided in addition to seat cushions or rigid surfaces allow proper spinal alignment has been shown to improve these problems. While numerous back support devices have been proposed in the prior art, these devices are single use devices and are often neglected or left behind as a user moves from one location to another. Numerous prior art devices are configured in a fixed configuration, such as a solid foam cylinder. There is a need for multi-purpose devices for lumbar and neck support which encourages a user to carry the support from one location to another.

US Publication 20030045824 discloses an inflatable portable lumbar support as encouragement to carry the support in a compact manner. The general idea of inflatable lumbar supports is found again in U.S. Pat. No. 3,974,827, where a longitudinal cavity is formed by inflation of a lumbar area of a user in a chair. Further encouraging portability of a lumbar support is one found in U.S. Pat. No. 4,471,993 for removable attachment by straps to the back of a chair, similar to the support found in U.S. Pat. No. 4,597,386. Also similar to the portable support of the '993 patent is one with an incorporated a very thin layered heating or cooling cell located just beneath a cover fabric. The '386 patent describes the necessity of making the cell very thin to accomplish the conforming function of the support from highly curved to entirely flat.

### SUMMARY OF THE INVENTION

The present invention is (1) a lumbar support and/or (2) a neck support incorporating a water or liquid containing bottle for beverages. The invention devices are shaped to provide lumbar or neck support but retain a removable cap and a large internal volume for storage of a beverage.

While prior art neck or lumbar supports have been proven to be effective for their intended purposes, they are sole purpose devices. In airplane or bus travel where possessions of a passenger within their reach is typically very limited, the passenger will typically carry a beverage bottle of some type and a lumbar or neck support. The present invention frees the passenger from having to carry two space taking devices and combines them into a single, easy to carry product incorporating in the form factor of the lumbar or neck support a beverage bottle.

It is an object of the invention that the beverage bottle incorporated into an encircling layer of padding material with an outside surface shape of the support surface and rear surfaces be capable of being removed and replaced through an opening in one of the end walls of the invention lumbar or neck support or by way of recloseable opening in the encircling padding material to replace a leaky or empty bottle with a full one. Beverage bottles may be relatively thin walled plastic well known in the field of non-carbonated and carbonated beverages or may be an insulated thermos or vacuum bottle that is insertable through an opening in an end wall of the invention lumbar or neck support.

Because re-filling the beverage bottle my result in spilling of liquids on the beverage bottle, an opening in or having removable padding material is another object of the invention relative to the beverage bottle.

Various objects and advantages of the present invention will become apparent from the following description taken in conjunction with the accompanying drawings wherein are set forth, by way of illustration and example, certain embodiments of this invention. The drawings submitted herewith constitute a part of this specification, include exemplary embodiments of the present invention, and illustrate various objects and features thereof.

### BRIEF DESCRIPTION OF THE DRAWINGS

FIG. 1 is a perspective view of a lumbar support device according to the invention.

FIGS. 2 and 3 are respectively front and rear views of the lumbar support device of FIG. 1.

FIGS. 4, 5, and 6 are respectively right, left and vertical section views of the lumbar support device of FIG. 1.

FIG. 7 is the lumbar support device of FIG. 4 shown in relation to seat cushions for use of the device for lumbar support.

FIG. 8 is a front view of a neck support device according to the invention.

FIGS. 9 and 10 are respectively right side and rear views of a neck support device according FIG. 8.

FIG. 11 is a vertical section view of the device of FIG. 9, showing an internal space for containing a beverage.

FIGS. 12 and 13 are respectively bottom and top views of the neck support device according FIG. 8.

### DETAILED DESCRIPTION OF THE INVENTION

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

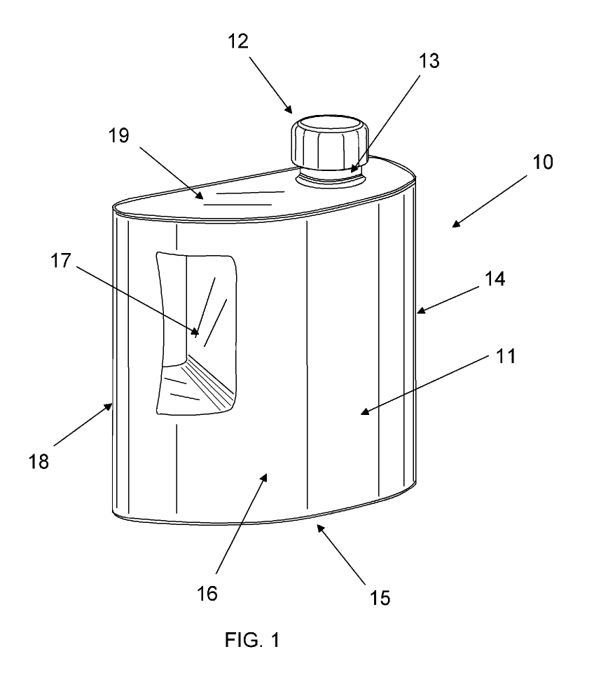

Referring to the drawings in more detail, FIG. 1 is a perspective view of a lumbar support device 10 according to the invention, comprising a lumbar support shaped bottle 11 defined by a convex front wall 16, a top edge 18, a bottom plate 15, a left side 15, and a right side 19 that supports a threaded cap 12 sealing an opening tube 13 for adding beverages to bottle 11 and for drinking them from bottle 11. Bottle 11 is understood to comprise a liquid tight and sealed container for beverages that a user will at some time drink from tube 13 after removing cap 12.

It is a critical feature of one embodiment of the inventions is for that cutout 17 be made in an upper part of bottle 11 so that a user may easily carry bottle 11 from one location to the next, as bottle 11 is intended to have substantial weight in the form of beverages contained in it. In a preferred embodiment, a slight downward curve is formed in the upper portion of cutout 17 to enhance support of the weight and to better conform to a human hand carrying the device.

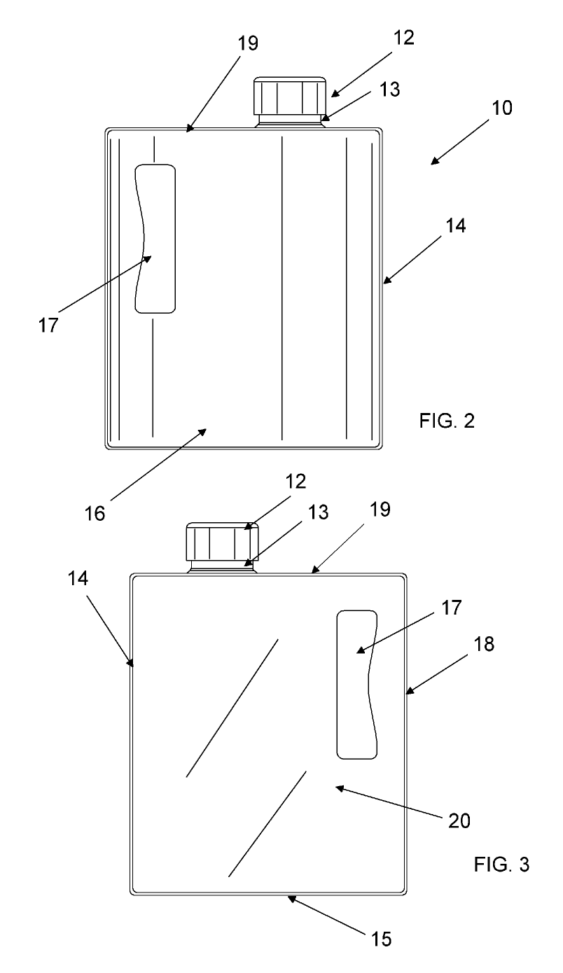

FIGS. 2 and 3 are respectively front and rear views of the lumbar support device 10 of FIG. 1.

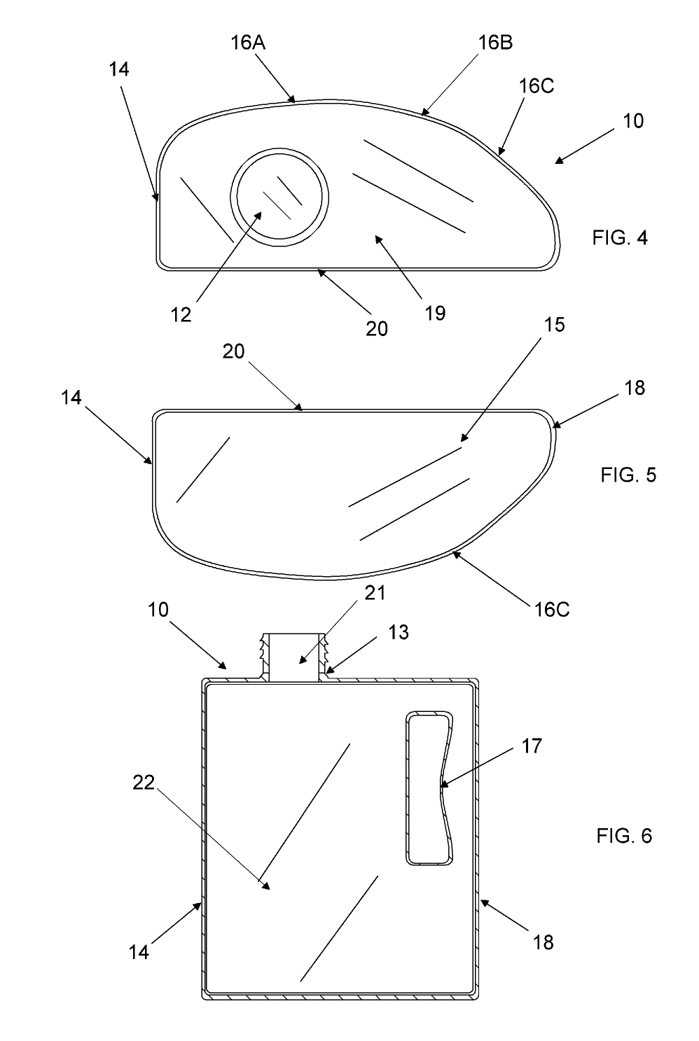

FIGS. 4, 5, and 6 are respectively right, left and vertical section views of the lumbar support device 10 of FIG. 1, showing a bore 21 in tube 13 that communicates between the internal, beverage containing space 21 and the outside of bottle 11.

FIG. 7 is the lumbar support device of FIG. 4 shown in relation to seat cushions for use of the device 10 for lumbar support, where a lumbar portion of a user's spine will contact one or more of surfaces 16a, 16b or 16c, urging the back plate 20 against a vertical cushion 23 and the bottom plate 14 against a horizontal cushion 24.

It is preferred that front plate 16 comprises flexible polymer material so that a user's lumbar spinal section may compress it instead of encountering a rigid support surface. It is an alternate embodiment of the device 10 that at least the front plate 11 be covered in compressible foam and a fabric surface to further enhance the comfort provided to a user of device 10.

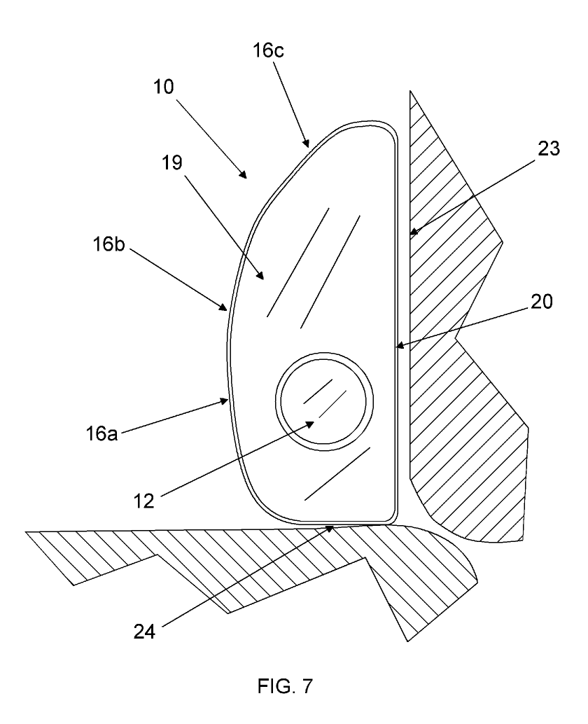

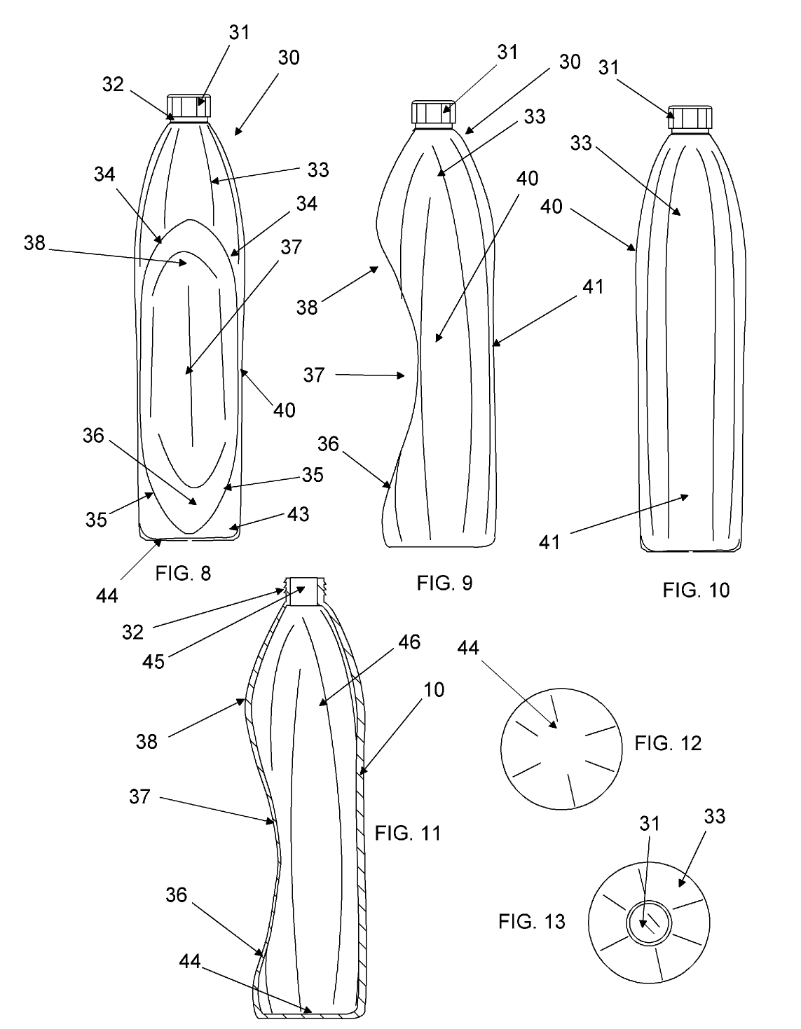

FIG. 8 is a front view of a neck support device 30 according to the invention, comprising in general a beverage bottle 33 and a cap 31 sealing a tube 32 that allows beverages to flow into and out of bottle 33.

Bottle 33 comprises a generally long vertical, cylindrical shape very similar to that of water or soft drink bottles, i.e., in the form factor of, say, the classic Coke® bottle in a one or two liter size. A user is more likely, thereby, to be accustomed to carrying a similar bottle, where the present device 30 comprises a neck support at impressions 36, 37 and 38 made in the general form factor of a beverage bottle. Impression 36 is generally outlined at lines 35 in a lower portion of bottle 30. Impression 38 is generally outlined at lines 34 in bottle 30. Impression 37 is continuous with and between impressions 36 and 38. The scalloped portion removed in the bottle form factor represented in bottle 30 is shallow at impressions 36 and 38 and deepest at impression 37 so that a the entire impression or scalloped section of bottle 30 is adapted to receive a base portion of a human neck so that bottle 30 is urged rearward of the neck pressing into impressions 36, 37 and 38, whereby a backside 41 is urged against a solid surface where a user's head might normally lie, resulting in elevation of the user's neck away from that solid surface and preventing the user's neck from being uncomfortably urged further toward that solid surface.

Bottle 30 comprises a sealed base 44 an right side 40, where lower section 43 is scooped out at impression 36 but is in the form factor of a cylindrical beverage bottle at backside 41.

FIGS. 9 and 10 are respectively right side and rear views of a neck support device 30 according FIG. 8.

FIG. 11 is a vertical section view of the device of FIG. 9, showing an internal space 46 for containing a beverage for communicating with the outside of bottle 30 by way of bore 45.

FIGS. 12 and 13 are respectively bottom and top views of the neck support device 30 according FIG. 8.

It is preferred that bottle 33 comprises flexible polymer material so that a user's neck may compress it instead of encountering a rigid support surface. It is an alternate embodiment of the device 30 that at least the impressions 36, 37 and 38 be covered in compressible foam and a fabric surface to further enhance the comfort provided to a user of device 30.

Thus, the invention in several embodiments is a portable lumbar support for support of a user's lower back in a chair or seat comprising a generally wedge shape of two end walls connected by a rear surfaces and a support surface. It will be appreciated from examination of the drawing figures that rear surfaces of the wedge shape are supported on a seat surface of the chair and a back surface of the chair, so that the support surface of the wedge shape is oriented upward and outward from the seat surface of the chair and the back surface of the chair to thereby support a back of a user. It is critical that a beverage bottle be incorporated within the wedge shape with a recloseable external opening of the bottle being accessible by a user. Padded material can entirely encircle of only partly encircle the support surface and rear surfaces. Thickness and softness of the padded materials depends upon a user's preference for soft or firm polymer foam or other padding materials and a cover fabric for the padded materials. For instance, a user may desire to be supported structurally almost entirely and not to sense the shape of the beverage bottle, in which case a beverage bottle has a small diameter relative to the entire cross section shape of the encircling padding materials and the beverage bottle is embedded more distant from the support surface and more toward the rear surfaces.

As described above, the overall shape of the invention portable neck support is in the form factor of a one or two liter soft drink bottle, but where the support surface is substantially flattened in a center portion and the rear surfaces comprise a generally cylindrical shape of the form factor of a one or two liter soft drink bottle.

The portable lumbar support of the invention can be a heating or cooling aid for the lower back or neck of a user, where all or a part of the support surface and rear surfaces are a minimally padded or no-padded layer hot or cold liquid contents of the beverage bottle transfer heat between a user and liquid contents of the beverage bottle through exposed surfaces of the beverage bottle so that hot or cold liquid contents of the beverage bottle heat or cool a user's back or neck.

It is to be understood that while certain forms of the present invention have been illustrated and described herein, it is not to be limited to the specific forms or arrangement of parts described and shown.

What is claimed is:

1. A portable lumbar support for support of a user's lower back in a chair or seat comprising a generally wedge shape of two end walls connected by a rear surfaces and a support surface, so that the rear surfaces of the wedge shape are supported on a seat surface of the chair and a back surface of the chair; the support surface of the wedge shape is oriented upward and outward from the seat surface of the chair and the back surface of the chair to thereby support a back of a user; and a beverage bottle is incorporated within the wedge shape with a recloseable external opening of the bottle being accessible by a user.

2. The portable lumbar support of claim 1 wherein the rear surfaces and the support surface comprise materials that partly or entirely cover outside walls of the beverage bottle.

3. The portable lumbar support of claim 2 wherein all or a part of the support surface is a padded layer adhered to an outer surface of the beverage bottle.

4. The portable lumbar support of claim 2 wherein the beverage bottle is a round or rectangular cross section shape and a padded layer surrounding the beverage bottle forms the wedge shape of the portable lumbar support.

5. The portable lumbar support of claim 1 wherein the support surface is outwardly convex.

6. The portable lumbar support of claim 1 wherein the support surface is substantially flattened.

7. The portable lumbar support of claim 6 wherein the support surface is substantially flattened in a center portion, substantially convex at ends of the portable lumbar support.

8. The portable lumbar support of claim 7 wherein the overall shape of the portable lumbar support is in the form factor of a one or two liter soft drink bottle, but where the support surface is substantially flattened in a center portion and the rear surfaces comprise a generally cylindrical shape of the form factor of a one or two liter soft drink bottle.

9. The portable lumbar support of claim 1 wherein all or a part of the support surface and rear surfaces are a padded layer adhered to all or a portion of the outer surface of the beverage bottle so that hot or cold liquid contents of the beverage bottle are insulated from sensation of heat or cold on a user's back or neck.

10. The portable lumbar support of claim 1 wherein all or a part of the support surface and rear surfaces are exposed surfaces of the beverage bottle so that hot or cold liquid contents of the beverage bottle heat or cool a user's back or neck.

11. The portable lumbar support of claim 1 wherein one of the two end walls has the external opening and a cutout is defined through the portable lumbar support so that a user may easily carry it.

12. A portable neck support for support of a user's neck against a back of a chair or seat comprising a cylindrical shape of two end walls connected by a rear surfaces and a support surface, so that the rear surfaces of the cylindrical shape are supported between a back surface of the chair and a user's neck; the support surface of the cylindrical shape is oriented forward from the surface of seat back thereby support a neck of a user; and a beverage bottle is incorporated within the cylindrical shape with a recloseable external opening of the bottle being accessible by a user.

13. The portable neck support of claim 12 wherein the rear surfaces and the support surface comprise materials that partly or entirely cover outside walls of the beverage bottle.

14. The portable neck support of claim 13 wherein all or a part of the support surface is a padded layer adhered to an outer surface of the beverage bottle.

15. The portable neck support of claim 13 wherein the beverage bottle is a round or rectangular cross section shape and a padded layer surrounding the beverage bottle forms the cylindrical shape of the portable lumbar support.

16. The portable neck support of claim 12 wherein the support surface is substantially flattened.

17. The portable neck support of claim 16 wherein the support surface is substantially flattened in a center portion, substantially convex at ends of the portable lumbar support.

18. The portable neck support of claim 7 wherein the overall shape of the portable lumbar support is in the form factor of a one or two liter soft drink bottle, but where the support surface is substantially flattened in a center portion and the rear surfaces comprise a generally cylindrical shape of the form factor of a one or two liter soft drink bottle.

19. The portable neck support of claim 12 wherein all or a part of the support surface and rear surfaces are a padded layer adhered to all or a portion of the outer surface of the beverage bottle so that hot or cold liquid contents of the beverage bottle are insulated from sensation of heat or cold on a user's back or neck.

20. The portable neck support of claim 12 wherein all or a part of the support surface and rear surfaces are exposed surfaces of the beverage bottle so that hot or cold liquid contents of the beverage bottle heat or cool a user's back or neck.
